(19) United States
(12) Patent Application Publication
(10) Pub. No.: US 2021/0299067 A1
(43) Pub. Date: Sep. 30, 2021

(54) GUANIDINIUM DERIVATIVES AS IMMUNITY-INDUCING AGENT

(71) Applicant: UCAR HEALTH GMBH, Munich (DE)

(72) Inventor: DILEK UCAR, Cesme/Lzmir (TR)

(21) Appl. No.: 17/251,532

(22) PCT Filed: Jun. 21, 2018
(86) PCT No.: PCT/TR2018/050314

§ 371 Date: Dec. 11, 2020

(51) Int. Cl. A61K 31/155; A61K 36/07; A61K 36/43; A61K 45/06; A61K 9/00; A61P 9/10; A61P 37/02

(52) U.S. Cl. CPC A61K 31/155; A61K 36/07; A61K 36/43; A61P 37/02; A61K 9/0021; A61P 9/10; A61K 45/06

(57) ABSTRACT

Use of an immune modulator composition and/or pharmaceutical composition comprising Guanidinium Derivatives substances for use in the manufacture of a medicament for the treatment of an autoimmune disease or autoimmune disorder, including certain vascular disorders. Immunity-inducing agent composition having a pH of between 5.5 and 7.5 containing at least 100 mg/l, preferably at least 300 mg/l of a combination food protecting agent and guanidinium derivatives, particularly to combinations of oligo(2-(2-ethoxy)ethoxy ethyl guanidinium chloride), poly(hexamethylendiamine guanidiniumchloride), polyetheramines, triethyleneglycol diamine, mushrooms, enzymes, PGPR, amino acids, antioxidants like humic acids and some natural products like phytotherapeutic plant extracts.

### TECHNICAL FIELD

The present invention relates to immune modulators, in particular vaccines that modulate a cellular immune response and uses thereof and present invention relates also to the use of immune modulators in the manufacture of a medicament for the treatment and/or prevention of an autoimmune disease or autoimmune disorder, including certain vascular disorders. This invention relates also to the field of immunotherapy composition. In immunotherapy, in order to reduce side effects, it is necessary that the peptide or protein to be recognized as the antigen exist hardly in normal cells and exist specifically in cancer cells. The present invention relates to a combination guanidinium derivatives, particularly to combinations of oligo(2-(2-ethoxy)ethoxy ethyl guanidinium chloride), poly(hexamethylendiamine guanidinium chloride), mushrooms, enzymes, PGPR, amino acids, antioxidants like humic acids and some natural products like phytotherapeutic plant extracts.

### PRIOR ART

Cancer is the commonest cause for death among all of the causes for death, and therapies carried out therefor at present are mainly surgical treatment, which may be carried out in combination with radiotherapy and/or chemotherapy. In spite of the developments of new surgical methods and discovery of new anti-cancer agents in recent years, treatment results of cancers have not been improved very much at present except for some cancers. In recent years, by virtue of the development in molecular biology and cancer immunology, cancer antigens recognized by cytotoxic T cells reactive with cancers, as well as the genes encoding the cancer antigens, were identified, and expectations for antigen-specific immunotherapies have been raised.

The present invention is predicated upon the surprising finding that Immunity-inducing agent administered to a test subject can elicit a modification of the immune system, in particular the cellular immune system of that test subject, which effects a preventative and/or therapeutic effect on autoimmune diseases or autoimmune disorders, particularly those which involve the inflammation of the intima of blood vessels for example.

A number of tumors such as mammary gland tumor and some of the cell carcinoma are known in pets such as dogs and cats, and they rank high also in the statistics of diseases in dogs and cats. However, at present, no therapeutic agent, synthesis agent or diagnostic agent exists which is effective for cancers in mouse, dogs and cats. Most of tumors in dogs and cats are realized by owners only after they advanced to grow bigger, and in many cases, it is already too late to visit a hospital to receive surgical excision of the tumor or administration of a human drug (an anticancer drug or the like), so that those dogs and cats often die shortly after the treatment. Under such circumstances, if therapeutic agents and prophylactic agents for cancer effective for dogs and cats become available, their uses for canine cancers are expected to be developed. Some of the protein related to cell cycle regulator involved in distribution of chromosomes, and reported to show higher expression in nasopharyngeal carcinoma, renal cancer, liver cancer and a certain type of breast cancer cells, compared to normal tissues (Patent Document 2, Non-patent Documents 3 to 5). It has been reported that the growth of cancer cells can be suppressed by suppressing expression of PDS5A in cancer cells using an antisense nucleic acid, ribozyme or siRNA against the PDS5A gene or using an antibody that specifically binds to the PDS5A protein, and that cancer cells can be induced to cause apoptosis by administering the full-length PDS5A protein or a partial peptide of the PDS5A protein (Patent Document 3). Further, in Patent Document 3, increase in the mRNA level of the PDS5A protein in cancer cells was confirmed. However, there is no report suggesting that the PDS5A protein and a partial peptide of the protein has an action to induce immunity against cancer cells and hence the protein and a partial peptide of the protein is useful for therapy or prophylaxis of cancer, and whether or not the PDS5A protein has a function as a marker that can be used for diagnosis of cancer has not been confirmed.

Ingredient of sentez Immunity-inducing agent was also humic substances. Humic substance occur mainly in heavily degraded peat. Humic acids (HAs) represent a group of high molecular-weight macromolecules consisting of complex polymeric aromatic structures. Together with fulvic acids, they represent certain fractions of the group of organic compounds called humic substances, which are by some considered inert and by others biotoxic. More detailed studies revealed controversial results: high doses of HA induced chromosomal abnormalities in intestinal cell via oxidative DNA damage, inhibited nuclear factor KB activation, and stimulated the thymus and neutrophils. On the other hand, several interesting and potentially clinically important biological activities were recently associated with various types of HA, including antiviral properties and proliferation of lymphocytes. Also, addition of HA into the feed of cultured animals results in improved growth and health. Concentrated peat extract is a natural immunomodulator. It is produced by peat. Briefly, production technology involves alkaline extraction of peat, acidification to remove humic acid fraction, and concentration connected with thermal processing. Scientific studies conducted by different research groups over many years confirmed biological activity of peat extract, but the exact mechanism of its action is still unclear. In particular, peat extract activates neutrophils, macrophages, synthesis of INF-β, INF-□ and TNF-□, as well as NK cells. Many researches also showed a remarkably low toxicity of the peat extract, the extract having no mutagenic, teratogenic or carcinogenic effects. It was used with good results in therapy of chronic disorders with inflammatory basis, for example respiratory tract infections.

An advantage of the use of compositions comprising Immunity-inducing agent to effectively treat and/or prevent autoimmune diseases and autoimmune disorders, particularly those which involve the inflammation of the intima of blood vessels for example, may be that this treatment and/or prevention is effected whilst producing fewer long-term side effects than the chemotherapies, i.e. the immunosuppressive medication, now routinely used.

The phrase “cellular immune system”, as used herein, includes a cell-mediated immune response which depends upon the presence of T lymphocytes. The term “T lymphocytes” includes cytotoxic T lymphocytes, helper T cells, suppresser T cells and regulatory T cells. Modification of a cell-mediated immune response may be used, for example, to overcome cell-mediated immune disorders including, for example autoimmune diseases or disorders.

The terms “modulate”, “modify”, “modification” and other derivatives thereof, as used herein, mean downregulating, inhibiting, inducing, stimulating, upregulating, altering or otherwise affecting a component or components of the cellular immune system.

The present inventors intensively studied to obtain a cDNA encoding a protein which binds to antibodies existing in the serum derived from a tumor-bearing living body, and, based on the cDNA, the canine PDS5 protein, a regulator of cohesion maintenance, homolog A, having the amino acid sequence Further, based on human and murine homologous genes of the obtained gene, human PDS5A having the amino acid corresponds to a partial sequence and murine PDS5A having the amino acid sequence. The present inventors then discovered that that these PDS5A are specifically expressed in tissues or cells of breast cancer, brain tumor, esophagus cancer, lung cancer, renal cancer, colon cancer, perianal adenocarcinoma, neuroblastoma and leukemia. Further, the present inventors discovered that, by administration of these PDS5A to a living body, immunocytes against PDS5A can be induced in the living body, and a tumor in the living body expressing PDS5A can be regressed. Further, the present inventors discovered that a recombinant vector which can express a polynucleotide encoding the full-length PDS5A protein or a fragment thereof can induce an anti-tumor effect against cancer expressing PDS5A in the living body.

Further, the present inventors discovered that a partial peptide of PDS5A has a capacity to be presented by antigen-presenting cells, thereby allowing activation and growth of cytotoxic T cells specific to the peptide (immunity inducing activity), and therefore that the peptide is useful for therapy and/or prophylaxis of cancer, and, further, that antigen-presenting cells which have contacted with the peptide and T cells which have contacted with the antigen presenting cells are useful for therapy and/or prophylaxis of cancer, thereby completing the present invention.

Thus, the present invention has the following characteristics.

An immunity-inducing agent comprising as an effective ingredient(s) at least one guanidin from oligo(2-(2-ethoxy)ethoxy ethyl guanidinium chloride), poly(hexamethylendiamine guanidiniumchloride), polyetheramines, triethyleneglycol diamine, having an immunity-inducing activity/activities, or as an effective ingredient(s) a recombinant vector(s) which comprise(s) a polynucleotide(s) encoding the polypeptide(s) and is/are capable of expressing the polypeptide(s) in vivo:

a. The immunity-inducing agent according to (1), wherein the guanidine (b) has a sequence identity of not less than 55% with amino acid. 20 types of amino acids constituting naturally occurring proteins may be classified into groups in each of which similar properties are shared, for example, into neutral amino acids with side chains having low polarity (Gly, Ile, Val, Leu, Ala, Met, Pro), neutral amino acids having hydrophilic side chains (Asn, Gln, Thr, Ser, Tyr, Cys), acidic amino acids (Asp, Glu), basic amino acids (Arg, Lys, His) and aromatic amino acids (Phe, Tyr, Trp). It is known that, in many cases, substitution of an amino acid within the same group doe not change the properties of the polypeptide. Therefore, in cases where an amino acid residue in the polypeptide (a) of the present invention is substituted, the probability that the immunity-inducing activity can be maintained may be increased by carrying out the substitution within the same group, which is preferred.

b. Guanidine-polypeptide having an immunity-inducing activity to a tumor-bearing living body enables regression of an already existing tumor. Therefore, the immunity-inducing agent of the present invention can be used as a therapeutic and/or prophylactic agent for cancer. Further, the Guanidine-polypeptide having an immunity-inducing activity can be used for a method of therapy and/or prophylaxis of cancer by immune induction.

c. The immunity-inducing agent according to (1), wherein each of the guanidine(s) having an immunity inducing activity/activities is a guanidine consisting essentially of not less amino acids in any one of the amino acid sequences

d. The immunity-inducing agent of the present invention may contain only a polypeptide or may be formulated by being mixed as appropriate with an additive such as a pharmaceutically acceptable carrier, diluent or vehicle suitable for each administration mode. Formulation methods and additives which may be used are well-known in the field of formulation of pharmaceuticals, and any of the methods and additives may be used. Specific examples of the additives include, but are not limited to, diluents such as physiological buffer solutions; vehicles such as sugar, lactose, corn starch, calcium phosphate, sorbitol and glycine; binders such as syrup, gelatin, gum arabic, sorbitol, polyvinyl chloride and tragacanth; and lubricants such as magnesium stearate, polyethylene glycol, talc and silica. Examples of the formulation include oral preparations such as tablets, capsules, granules, powders and syrups; and parenteral preparations such as inhalants, injection solutions, suppositories and solutions. These formulations may be prepared by commonly known production methods

e. The immunity-inducing agent of the present invention may be used in combination with an immunoenhancer capable of enhancing the immune response in a living body. The immunoenhancer may be contained in the immunity-inducing agent of the present invention or administered as a separate composition to a patient in combination with the immunity-inducing agent of the present invention.

f. By culturing the antigen-presenting cells in the coexistence of the above-described guanidine-polypeptide, the guanidine-polypeptide is incorporated into MHC molecules of the antigen-presenting cells and presented on the surface of the antigen-presenting cells. Therefore, using the above-described guanidine-polypeptide, isolated antigen-presenting cells containing the complex between the guanidine-polypeptide and the MHC molecule can be prepared. Such antigen-presenting cells can present the guanidine-polypeptide against T cells in vivo or in vitro, to induce, and allow proliferation of, cytotoxic T cells specific to the polypeptide. The immunity-inducing agent according to any one of for prophylaxis of a cancer in an animal.

g. The immunity-inducing agent according to (a) or (c), for therapy of a cancer in an animal.

h. The immunity-inducing agent according to (a) or (d), wherein the cancer is a cancer expressing PDS5A.

i. The immunity-inducing agent according to any one of (a) to (e), wherein the cancer is breast cancer, brain tumor, esophagus cancer, lung cancer, renal cancer, colon cancer, perianal adenocarcinoma, neuroblastoma or leukemia.

j. The immunity-inducing agent according to any one of (a) to (f), further comprising an immunoenhancer

There is thus a tendency to look for harmless alternatives which can be used immunity-inducing agent. Amongst these, spices and/or extracts from various plants or fruits have proven to be effective antimicrobial agents. For instance, Weiss J. et al. in Journal of Food Protection, Vol. 68, No. 12, 2005, p. 2559-2566 and in Journal of Food Protection, vol. 68, No. 7, 2005, p. 1359-1366 describe the antimicrobial effect of essential oil components.

U.S. Pat. No. 4,710,387 issued on Dec. 1, 1987, to Dirk J. D. Uiterwaal et al. describes a nutritional supplement preparation for pregnant and breast-feeding women based on milk constituents and the process of preparation. The composition contains by weight 10-20% protein, 16-28% fat, 43-65% carbohydrates, at most 3.5% moisture, minerals, trace elements, and vitamins such as calcium, phosphorus, magnesium, copper, zinc, iodine, iron, vitamins A, B1, B6, C, D3, E, niacin, folic acid, and optionally flavoring and/or colorant. The composition is distinguishable for being tailored to supply nutrients to a pregnant or breast-feeding woman and requires large amounts of fat (linoleic acid) and carbohydrates (lactose, dextrins and sucrose).

U.S. Pat. No. 5,770,217 issued on Jun. 23, 1998, to Frank J. Kutilek, III et al. describes a dietary pilled supplement comprising herbs, herbal extracts, vitamins, minerals, and amino acids effective in modulating hematological toxicities, enhancing the immune system and maintaining appetite and weight. The supplement contains a large amount of crucifer extract (8-12 wt. %) and ascorbic acid or vitamin C (8-13 wt. %). The amino acids include glutathione, L-cysteine and L-methionine. The composition is distinguishable for requiring crucifer extract and amino acids in pill form.

U.S. Pat. No. 4,348,379 issued on Sep. 7, 1982, to Horst Kowalsky et al. describes a dietetic composition for natural digestion regulation comprising in parts by weight each of 50-150 of whole fleawort seeds, whole linseed, wheat bran, and lactose. A binding agent based on natural rubber and optionally, flavor and/or food color. The composition is distinguishable for requiring fleawort seeds and whole linseed.

U.S. Pat. No. 4,440,760 issued on Apr. 3, 1984, to Rex E. Newnham describes a food supplement for the relief of arthritic conditions comprising in parts by weight of 2-500 of sodium tetraborate, 150 each of the dried herbs Gauaiacum, Berberis and Harpagophytum, 1 ppm Rhus-tox and/or Bryonia, gum arabic as binder, starch as a disintegration aid, and magnesium stearate as a lubricating aid in tablet forming. The composition is distinguishable for requiring sodium tetraborate and several dried herbs not required in the present invention.

U.S. Pat. No. 5,332,579 issued on Jul. 26, 1994, to Anthony J. Umbdenstock describes a nutritional supplement for optimizing cellular health of recovering drug addicts, alcoholics, smokers, etc., comprising: 1,500-15,000 I.U. vitamin A; 5,000-45,000 I.U. Beta-carotene; 33-300 mg. vitamin B1; 50-1,000 mg. vitamin B6; 30-300 mcg. vitamin B12; 20-500 mg. niacin; 100-2,000 mg. niacinamide; 100 mg. vitamin C; 5-100 mg. magnesium; 10-100 mg. zinc; 50-1,000 mg. valerian root; at least two minerals selected from the group consisting of calcium, 20-500 mcg. chromium, copper, iron, 5-1,000 mg. manganese, and selenium; and at least four additional vitamins, herbs, and amino acids selected from the group consisting of 100-1,000 I.U. vitamin D3, 10-800 I.U. vitamin E, 5-100 mg. vitamin B2, 100-1,000 mcg. biotin, 50-500 mg. pantothenic acid, 70-900 mg. choline, 100-1,000 mg. inositol, 50-1,000 mg. glutamic acid, 50-1,000 mg. glutamine, and echinachea. The composition is distinguishable for omitting beans, peas, berries, and grains. U.S. Pat. No. 5,656,312 issued on Aug. 12, 1997, and U.S. Pat. No. 5,834,048 issued on Nov. 10, 1998, to Udo Erasmus et al. describes a daily dietary food supplement composition packaged in a sealed pouch for humans comprising at least by weight proportions, 71-73% flax seeds, 5% yeast, 6% rice and bran yeast, 2% liver, 2% alfalfa, 1% bone, 2% carrot, 2% apple, 0.07% kelp. 0.01% lecithin, 0.01% garlic, 0.02% taurine, 0.01% equiteum herb, and 0.01% carnitine. The composition must be prepared at a temperature below 100° F. for less than 20 minutes and in the limited illumination of red light. The food composition is distinguishable for requiring liver, yeast, flax seeds, garlic, and taurine as well as limited heating and lighting conditions.

U.S. Pat. No. 5,925,377 issued on Jul. 20, 1999, to Teja D. Gerth et al. describes a dietary supplement composition combining amino acids, minerals, herbs, vitamins, diuretics, and digestive enzymes. For example, D,L-phenylalanine is combined with tyrosine, L-glutamine and St. John's wort to act as an appetite depressant while L-carnitine is combined with chromium picolinate to work as fat directors to convert stored body fat into energy. The composition is distinguishable for requiring diuretics and digestive enzymes.

U.S. Pat. No. 5,976,579 issued on Nov. 2, 1999, and U.S. Pat. No. 6,143,332 issued on Nov. 7, 2000, to Linsey McLean describes a nutritional supplement for the prevention and treatment of excessive intestinal permeability comprising at least 50 wt. % nutritional buffers (calcium carbonate), amino acid chelates (selenium, copper, zinc, manganese, iodine, and chromium), minerals, vitamins (A, B-complex, D, and E), antioxidants, free radical scavengers, and intestinal tract-soothing herbs. The composition is distinguishable for requiring buffers, chelates, antioxidants, and free radical scavengers.

U.S. Pat. No. 6,238,672 B1 issued on May 29, 2001, to Jau-Fei Chen describes dietary supplements containing dehydrated cactus fruit juice and ginseng berry juice for food products, drinks, capsules, and tablets. The supplement is distinguishable for requiring cactus fruit juice and ginseng berry juice for immunity-inducing agent.

U.S. Pat. No. 6,264,995 issued on Jul. 24, 2001, to Thomas Newmark et al. describes a herbal composition for reducing inflammation in bones and joints comprising holy basil, tumeric, ginger, green tea, rosemary, huzhang, Chinese goldthread, barberry, oregano, and scutellariae baicalensis. The composition is distinguishable for its medicinal characteristics and for immunity-inducing agent.

German Patent Application No. DE 31 43 926 A1 published on May 11, 1983, for Kurt Jesselring et al. describes a dietetic composition containing bran and/or pectin, vitamins, minerals, customary auxiliaries and carriers, and an anti-thrombotically active fraction derived from Basidiomycetes such as Auricularia, Himeola auricula judae, Polyporus ovinus, Polyporus giganteus, and Sparassis crispa. The composition is distinguishable for requiring an antithrombotically active fraction.

German Patent Application No. DE 44 16 402 A1 published on Nov. 30, 1995, for Harro Carstens et al. describes an immunity improving immunity-inducing agent comprising ethanolic extra extracts of medicinal herbs (aloe), vegetable oils containing eugenol, and, optionally, conventional stabilizers and additives. The medicinal herb extracts have a detoxifying effect through the stomach and intestinal tract. The composition is distinguishable for requiring only herb extracts, vegetable oils and eugenol.

French Patent Application No. 2 737 849 published on Feb. 21, 1997, for Jean P. Curtay et al. describes an orally administered food supplement for adults over forty years of age comprising: (1) an excipient (gum arabic or starch); (2) mineral salts (calcium carbonate, magnesium carbonate, zinc citrate; (3) vitamins B1, B2, B6, B8, B9, B12, C, E, and PP; (4) beta-carotene, (5) borage oil (herb); (6) fish oil; and (7) methionine. The composition is distinguishable for requiring beta-carotene, borage oil and methionine.

German Patent Application No. DE 196 53 100 A1 published on Jul. 23, 1998, for Adolph Metz describes a lactose-containing magnetic capsule food supplement comprising: (1) ferromagnetic magnetite; (2) piezoelectric rock crystal (silica); (3) magnesite powder (magnesium carbonate); (4) ginseng root, taiga root, mistletoe, ginkgo biloba leaves, hawthorn flowers or leaves, horse chestnut leaves, milk thistle, balm mint leaves, St. John's wort, speedwell, linden flowers, arnica flowers, lesser centaury (Erythraea centaurium), marigold flowers, yarrow (Achillea millefolium), red soapwort, and calamus root; and (5) vitamins A, C, E, aneurin, riboflavin, pyridoxine, B12, and Q10; reduced glutathione, glutamine, cysteine, methionine; (6) Ca-, Mg- and K-citrate; (7) E. coli or Lactobacillus acidophilus; (8) heartwood of Thuaja plicata; (9) oak bark; (10) aspirin and/or willow bark; (11) zinc, selenium and manganese; and (12) lactose, starch and dextrose. The composition is distinguishable for requiring a vast variety of exotic herbs, vitamins, lactose, minerals, and a magnetic constituent.

W.I.P.O. Patent Application No. WO 98/00024 published on Jan. 8, 1998, and W.I.P.O. Patent Application No. WO 98/47376 published on Oct. 29, 1998, for Houn S. Hsia describes a diet supplement composition to increase the level of high density lipoprotein (HDL) and calcium ions, and to decrease the levels of free radicals and glucose in human blood plasma comprising; (1) anti-oxidants selenium, vitamins A, B, C, D, and E, and fruit or vegetable juice concentrates; (2) green barley composition; (3) tincture of ginkgo biloba extract; and (4) minerals. The composition is distinguishable for requiring minerals, ginkgo biloba, and fruit and vegetable juice concentrates.

German Patent Application No. DE 199 07 586 A1 published on Aug. 24, 2000, for Waldemar Braun et al. describes a daily nutritional composition comprising (a) a basic kit for constant circadian dosage combined with (b) an “add-on” supplement used in time-dependent amounts. The basic kit contains specific amounts of various vitamins and minerals including beta-carotene, vitamins B1, B2, B6, B12, niacin, pantothenic acid, biotin, folic acid, phylloquinone, calcium, magnesium, manganese, zinc, iron, selenium, chromium, molybdenum, copper, and iodine. The “add-on” composition contains apple vinegar powder, artichoke extract, carnitine, guarina, silica, creatine, lecithin, and taurine. The compositions are distinguishable for requiring minerals and the “add-on” composition.

Herbal medicine has been in use for centuries by people of Asia and Europe. In the United States (US), herbs have become commercially valuable in the dietary supplement industry as well as in holistic medicine. Approximately one third of the US population has tried some form of alternative medicine at least once (Eisenberg et al., N. Engl. J. Med., 328:246-252 (1993)). Botanicals have also become a focal point for the identification of new active agents to treat diseases. Active compounds, derived from plant extracts, are of continuing interest to the pharmaceutical industry. For example, taxol an antineoplastic drug obtained from the bark of the western yew tree, has been found to be useful in the treatment of breast cancer (Gomez-Espuch et al., Bone Marrow Transplant, 25(3):231-235 (2000)).

There are many branches of herbal medicine around the world, such as Ayurveda, Unani, Sida and Traditional Chinese medicine (TCM). While modern Western medicine typically consists of administering a single chemical entity capable of intervening a specific biochemical pathway, each formula of TCM contains hundreds of chemical entities from several herbs which are designed to interact with multiple targets in the body in a coordinated manner. Although empirical practice contributed in a significant way to the herbal composition and prescription of these ancient herbal medicines, they are also supported, to a varying degree, by a set of theories which all are distinct from that of modern Western medicine in terms of anatomy, pharmacology, pathology, diagnosis treatment, etc. Among the different herbal medicine fields, TCM has developed a more complete set of theories over several centuries which have been well documented and practiced by local physicians caring for a huge population (>1.3 billion people) in greater China and in East Asia including Korea and Japan

Mixtures of botanical extracts, rather than a single compound are widely used throughout the world for the management of disease and are slowly gaining increased acceptance in Western countries (Okada, F., Lancet 348: 5-6 (1996); Xiao P G, Xing S T and Wang L W, Journal of Ethnopharmacol 38: 167-175 (1993)). The use of Traditional Chinese medicine is based on the interaction of many chemical components in an herbal preparation that act simultaneously and synergistically on multiple molecular targets and cellular mechanisms. These components serve various functions; some may be responsible for efficacy while others may decrease toxicity or increase bioavailability. Chinese herbal formulations are perhaps the best known botanical drugs and have been derived from empiric observations in humans over the millennia. The claimed indication of a given Chinese medicinal preparation, in many cases, is multiple rather than single. This is not surprising, due to the many phyto-chemical ingredients in a formulation that could exert actions at multiple targets. It is possible that one Chinese medicinal formulation may relieve more than one side effect associated with the use of cancer chemotherapeutic agents.

### THE PURPOSES AND BRIEF DESCRIPTION OF THE INVENTION

### Detailed Description of the Invention

The present invention is directed to immunity-inducing agent compositions containing a wide variety oligo(2-(2-ethoxy)ethoxy ethyl guanidinium chloride), poly(hexamethylendiamine guanidinium chloride), mushrooms, enzymes, PGPR, amino acids, antioxidants like humic acids and some natural products like phytotherapeutic plant extracts vitamins, herbs, and spices in the physical form of a powder mixture.

The groups of ingredients will be in terms of (1) oligo(2-(2-ethoxy)ethoxy ethyl guanidinium chloride), poly(hexamethylendiamine guanidinium chloride), (2) mushrooms, enzymes, PGPR, amino acids, antioxidants, (3) multiple vitamins, minerals and calcium, and (4) herbs and spices.

(1) A combination guanidinium derivatives, particularly to combinations of oligo(2-(2-ethoxy)ethoxy ethyl guanidinium chloride), poly(hexamethylendiamine guanidinium chloride), mushrooms, enzymes, PGPR, amino acids, antioxidants like humic acids and some natural products like phytotherapeutic plant extracts. The beans will include all of the following itemized twelve beans and peas (some with dual names) in equal amounts, such as in teaspoons, for a bean batch: red kidney bean, pinto bean (mottled kidney bean), black-eyed pea (cowpea), navy bean (white-seeded kidney bean), lima bean, green split pea, lentil seed, Turkish bean, yellow split pea, garbanzo bean (chick pea), black bean, and azuki beans (brown, Japanese). Therefore, 12 teaspoons of beans will be added.

(2) A combination guanidinium derivatives, particularly to combinations of oligo(2-(2-ethoxy)ethoxy ethyl guanidinium chloride), poly(hexamethylendiamine guanidinium chloride), mushrooms, enzymes, PGPR, amino acids, antioxidants like humic acids and some natural products like phytotherapeutic plant extracts. The bread grains include all of the following itemized grains added in equal amounts such as in teaspoons for a grain batch: oat bran, raw buckwheat groat (no hull), buckwheat hull, steel oats, rye flakes, wheat berry, barley wheat bran, whole millet, kamut, whole puinoa, wheat bran, and spelt flakes. Therefore, thirteen teaspoons of grains will be added.

(3) This group will contain multiple vitamins, minerals and calcium in terms of vitamin pills containing these ingredients such as: naturally occurring proteins, vitamins (A, B-12, C, D, E, and K), niacin (nicotinic acid), thiamine (vitamin B-1), biotin and pantothenic acid (vitamin B complexes), calcium, iron, iodine, magnesium, phosphorous, zinc, selenium, copper, potassium, molybdenum, manganese, and chromium. The quantities added of each ingredient will be based on the recommended daily dosage.

(4) The herbs and spices are added in small amounts such as teaspoons each of: pure oregano, mint, rosemary, basil, anise seed, fennel seed, garlic, sea salt, red pepper, thyme, parsley, clover, nutmeg, cinnamon, and cloves. However, the spices will be adjusted in terms of mild spice composition, medium spice composition, and hot spice composition for each of the following compositions.

(5) By the present invention, a novel immunity-inducing agent useful for therapy, prophylaxis and/or the like of cancer is provided. As concretely described in the late-mentioned Examples, administration of the guanidine base material used in the present invention to a living body enables induction of immunocytes in the living body, and a cancer which has already occurred can be reduced or regressed. Therefore, the guanidine base material is useful for therapy and/or prophylaxis of cancer.

The herbal compositions of the present invention are particularly useful with antiviral therapies. Preferably, the herbal compositions are administered with antiviral agents useful for treating AIDS. More preferably, the herbal compositions are administered with antiviral agents selected from the group consisting of AZT, D4T, DDI, 3TC, ddC, and FTG.

### Detailed Aspects of the Present Invention

The present invention further provides the use of an immune modulator composition or a pharmaceutical composition comprising Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts the manufacture of a medicament for the treatment or prevention of an autoimmune disease or an autoimmune disorder.

Suitably, the autoimmune disease or autoimmune disorder is of the type where the subjects own immune system damages one or more of the subjects tissues. Suitably, the autoimmune response may be triggered by something within the subject or something within the subject's environment. The autoimmune disease or autoimmune disorder according to the present invention may be one which follows an initiating cause. For example, the autoimmune disease or autoimmune disorder according to the present invention may be one which is caused by an infection and/or some other initiating cause. Potential initiating causes may be, by way of example, old age, infection (for example parasitic infection), treatment with steroids, repeated vaccination with alum, pregnancy and/or cancers.

The present invention further provides the use of an immune modulator composition or a pharmaceutical composition comprising Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts in the manufacture of a medicament for the treatment or prevention of an autoimmune disease, wherein the autoimmune disease or autoimmune disorder involves inflammation of the intima of a blood vessel. The autoimmune disease or autoimmune disorder according to the present invention may, as well as involving inflammation of the intima of a blood vessel, involve inflammation of the muscular layer of a blood vessel or of the myocardium. The autoimmune disease or autoimmune disorder according to the present invention may be one which is preceded by or caused by a vascular disorder.

The autoimmune disease or autoimmune disorder according to the present invention may be one or more of the following: arthritis, particularly rheumatoid arthritis, psoriasis, psoriatic arthropathy, scleroderma, thyroiditis, post-transplant intimal hyperplasia, graft rejection and vascular disorders.

The vascular disorders according to the present invention may include any vascular disease or disorder which comprises an autoimmune element, for example one which is caused by an autoimmune response. Vascular disorders according to the present invention may include one or more of Raynaud's disease and phenomenon, anterior uveitis, obliterative vascular disorder, atheroma formation (otherwise known as arteriosclerosis), arteritis, myointimal hyperplasia (natural or following angioplasty), inflammatory and autoimmune thickening of the intima and/or muscular layer of blood vessels, inflammatory blood vessel lesions, atherosclerotic heart disease, reperfusion injury, cardiac conduction disturbances, myocarditis, myocardial infarction.

The graft rejection according to the present invention may be chronic graft rejection, particularly in the absence of an immunosuppressant. Thus, the composition according to the present invention may be used as a replacement for the conventional immunosuppressant administered prior to, during and/or after transplantation. The compositions according to the present invention may be used when transplanting natural or artificial cells, tissues and organs, such as one or more of the following: corneas, bone marrow, organs (e.g. kidney, liver), lenses, pacemakers, natural or artificial skin tissue, islet cells. The present invention further provides the use of an immune modulator composition or a pharmaceutical composition comprising humic substances in the manufacture of a medicament for the treatment or prevention of a vascular disorder.

The present invention further provides the use of an immune modulator composition or a pharmaceutical composition comprising Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts in the manufacture of a medicament for the treatment or prevention of arthritis, particularly rheumatoid arthritis.

The present invention further provides the use of an immune modulator composition or a pharmaceutical composition comprising Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts the manufacture of a medicament for the treatment or prevention of graft rejection.

In a further aspect the present invention provides the use of an immune modulator composition or a pharmaceutical composition comprising Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts in the manufacture of a medicament for the treatment or prevention of psoriasis.

The term “immune modulator”, as used herein, means a substance which modulates a cellular immune system of a subject.

The term “whole cell”, as used herein, means a bacterium which is intact, or substantially intact. In particular, the term “intact” as used herein means a bacterium which is comprised of all of the components present in a whole cell, particularly a whole, viable cell, and/or a bacterium which has not been specifically treated to remove one or more components from it. By the term “substantially intact” as used herein it is meant that although the isolation and/or purification process used in obtaining the bacterium may result in, for example, a slight modification to the cell and/or in the removal of one or more of the components of the cell, the degree to which such a modification and/or removal occurs is insignificant. In particular, a substantially intact cell according to the present invention has not been specifically treated to remove one or more components from it.

Although it has been suggested that individual components of bacterial cells could be used to elicit an adjuvant effect, prior to the present invention the use of Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts in accordance with the present invention was not contemplated. Surprisingly, it has been found that by using humic substances in treatment and/or prevention of an autoimmune disease or an autoimmune disorder can be effected. The modulation of a cellular immune response caused by administration of humic substances may be advantageously long lasting as compared with the response elicited by administration of an individual component of the bacterium.

Preferably, the composition according to the present invention comprises more than one whole cell, and more preferably comprises a plurality of whole cells. The immune modulator composition according to the present invention may comprise an antigen and an adjuvant, wherein said adjuvant comprises humic substances. In another aspect, the immune modulator composition may be a pharmaceutical composition comprising Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts and optionally a pharmaceutically acceptable carrier, diluent or excipient, which immune modulator composition in use modifies a cellular immune response.

In a further aspect, the immune modulator composition and/or a pharmaceutical composition may comprise humic substances and at least one added cytokine, such as interleukin 2 for example. The cytokine may help to reinforce the immune modulatory action of the present invention.

The immune modulator composition and/or pharmaceutical composition may comprise two or more such antigens or antigenic determinants. In a further aspect, the immune modulator composition or pharmaceutical composition comprising Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts according to the present invention may be used in or as a vaccine. The vaccine may be a prophylactic vaccine or a therapeutic vaccine.

In a further aspect, the present invention provides an immune modulator composition or a pharmaceutical composition comprising Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts for use in the treatment or prevention of an autoimmune disease or an autoimmune disorder. In one aspect, humic substances according to the present invention may downregulate a Th2 response. In another aspect, the whole cell of the bacterium according to the present invention may upregulate a Th1 response. Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts may downregulate a Th2 response and upregulate a Th1 response. Alternatively, Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts upregulate a Th1 response whilst not affecting a Th2 response. Alternatively, Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts may downregulate a Th2 response, whilst also downregulating a Th1 response. Humic substances may upregulate a Th2 response, whilst also upregulating a Th1 response.

In another aspect, the present invention provides a method for treating or preventing an autoimmune disease or an autoimmune disorder comprising administering an effective amount of a pharmaceutical composition and/or immune modulator composition comprising Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts to a subject wherein the said composition modulates a cellular immune response.

The effective amount of the pharmaceutical composition and/or immune modulator composition may be administered as a single dose. Alternatively, the effective amount of the pharmaceutical composition and/or immune modulator composition may be administered in multiple (repeat) doses, for example two or more, three or more, four or more, five or more, ten or more, or twenty or more repeat doses. In particular, such repeated doses may be needed for the treatment of established and chronic conditions, for example. In a further aspect of the present invention, there is provided a method for protecting, including immunizing, a subject against an autoimmune disease or an autoimmune disorder comprising administering a pharmaceutical composition and/or immune modulator composition comprising Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts

The term “protected” as used herein means that the subject is less susceptible to the disease/disorder as compared with a subject not treated or administered with the compositions according to the present invention and/or that the subject is more able to counter or overcome the disease/disorder as compared with a subject not treated or administered with the compositions according to the present invention.

In another aspect, the present invention provides administering an effective amount of a pharmaceutical composition and/or an immune modulator composition according to the present invention to a subject, wherein said composition is co-administered with an antigen or antigenic determinant. Preferably, a medicament according to the present invention is used for the treatment or prevention of an autoimmune disease or an autoimmune disorder.

In a further aspect of the present invention, the pharmaceutical composition or the immune modulator composition according to the present invention may comprise Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts.

The term “subject”, as used herein, means an animal. Suitably, the subject may be for example any animal, including birds, crustaceans (such as shrimps for example), fish and mammals. Preferably, the subject is a mammal, including for example livestock and humans. In some aspects of the present invention, the subject may suitably be a human.

When the pharmaceutical composition or immune modulator composition is administered (for the first time if more than one administration is to be made) to livestock, preferably it is administered after the livestock has suckled for the first time. In particular, for some applications it may be important to allow the infant to take in and/or digest the parents colostrum prior to administering the first (where there is more than one dose) or only dose of the pharmaceutical composition or immune modulator composition. For the avoidance of doubt, for some applications the first administration of the pharmaceutical composition or immune modulator composition should be given between about 1-4 days post-birth, preferably 1-3 days post-birth, more preferably 1-2 days post-birth, preferably 2-3 days post-birth. Subsequent administrations may be given 7 days and/or 8-12 weeks after the first injection.

The term “immune modulator” as used herein includes a vaccine.

### Therapeutic Uses

The immune modulators of the present invention may be used in therapy. In particular such compounds may be used to modulate T lymphocyte responses in vivo and/or other cells involved in an immune response in vivo.

Immune modulator/pharmaceutical compositions capable of modulating, in particular blocking, T cell proliferation and/or differentiation and/or activity may be used against any disorder which is susceptible to prevention or treatment by the modulation of an adaptive immune response, i.e. a cellular immune response.

The autoimmune disease or autoimmune disorder according to the present invention is of the type where the subjects own immune system damages one or more of the subjects tissues. Suitably, the autoimmune response may be triggered by something within the subject or something within the subject's environment. The autoimmune disease or autoimmune disorder according to the present invention may be one which follows an initiating cause. For example, the autoimmune disease or autoimmune disorder according to the present invention may be one which is caused by an infection and/or some other initiating cause. Potential initiating causes may be, by way of example, old age, infection (for example parasitic infection), treatment with steroids, repeated vaccination with alum, pregnancy and/or cancers. The autoimmune disease or autoimmune disorder according to the present invention may be one which involves inflammation of the intima of a blood vessel. The autoimmune disease or autoimmune disorder according to the present invention may, as well as involving inflammation of the intima of a blood vessel, involve inflammation of the muscular layer of a blood vessel or of the myocardium. The autoimmune disease or autoimmune disorder according to the present invention may be one which is preceded by or caused by a vascular disorder.

The autoimmune disease or autoimmune disorder according to the present invention may be one or more of the following: arthritis, particularly rheumatoid arthritis, psoriasis, psoriatic arthropathy, scleroderma, thyroiditis, post-transplant intimal hyperplasia, graft rejection and vascular disorders. The vascular disorders according to the present invention may include any vascular disease or disorder which comprises an autoimmune element, for example one which is caused by an autoimmune response. Vascular disorders according to the present invention may include one or more of Raynaud's disease and phenomenon, anterior uveitis, obliterative vascular disorder, atheroma formation (otherwise known as arteriosclerosis), arteritis, myointimal hyperplasia (natural or following angioplasty), inflammatory and autoimmune thickening of the intima and/or muscular layer of blood vessels, inflammatory blood vessel lesions, atherosclerotic heart disease, reperfusion injury, cardiac conduction disturbances, myocarditis and myocardial infarction.

The graft rejection according to the present invention may be chronic graft rejection, particularly in the absence of an immunosuppressant. Thus, the composition according to the present invention may be used as a replacement to the conventional immunosuppressant administered prior to, during and/or after transplantation. The compositions according to the present invention may be used when transplanting natural or artificial cells, tissues and organs, such as one or more of the following: corneas, bone marrow, organs (e.g. kidney, liver), lenses, pacemakers, natural or artificial skin tissue, islet cells.

### T Helper Cells

The term ‘Th1’ as used herein refers to a type 1 T-helper cell (Th1). The term may also be used herein to refer to the response mediated by or through such a cell type. Such a response may include one or more of the secretion of Interleukin-2 (IL-2), the secretion of Interferon-gamma (IFN-γ), activation of macrophages, activation of cytotoxic T-cells, or any other Th1-associated event. Thus, the term ‘Th1’ may include Th1 cell(s) as well as the immune response(s) which such cell(s) produce.

The term ‘Th2’ as used herein refers to a type 2 T-helper cell (Th2). The term may also be used herein to refer to the response mediated by or through such a cell type. Such a response may include one or more of the secretion of Interleukin-4 (IL-4), the secretion of the splice variant interleukin IL-452, the secretion of Interleukin-5 (IL-5), increase in levels of cell determinant 30 (CD30) on lymphocytes, increase in levels of Immunoglobulin-E (IgE) in the blood or eosinophils in the blood, or any other Th2-associated event. Thus, the term ‘Th2’ may include Th2 cell(s) as well as the immune response(s) which such cell(s) produce.

It is known that various conditions may result in or from an unregulated or inappropriately regulated cellular immune response, in particular in the activation and/or proliferation of Th1 and/or Th2, which if left unregulated or inappropriately regulated has been found to result in one or more detrimental effects on the subject.

An unregulated or inappropriately regulated cellular immune response has also been observed in autoimmune disorders such as for example inflammatory vascular diseases such as arteriosclerosis, myointimal hyperplasia following angioplasty and anterior uveitis, and during graft transplantation/rejection. By way of further example, Stansby et al. Eur J Vasc Endovasc Surg 2002; 23: 23-28 tested the hypothesis that treatment with a mycobacterial preparation that modulates the antibody response, would diminish vascular disease, i.e. restenosis in a rat angioplasty model. It was shown that immunomodulation with mycobacterial material suitable for use in man, can reduce MIH. Since such modulation has low risk, this raises the prospect of an important new therapeutic modality to combat restenosis.

Accordingly, an aim of the present invention is to promote and establish the regulation of a cellular immune response, including the regulation or modulation of Th1 and/or Th2, in such a way so as to overcome the negative effects of the unregulated or inappropriately regulated cellular immune response. The use of an immune modulator composition and/or pharmaceutical composition according to the present invention modulates the Th1 or Th2 response, i.e. a Th1 or Th2 response that results in, for example, tissue damage. The use of an immune modulator composition and/or pharmaceutical composition according to the present invention may decrease the Th1 response and decrease the Th2 response. The use of an immune modulator composition and/or pharmaceutical composition according to the present invention may increase the Th1 response without affecting the Th2 response. The use of an immune modulator composition and/or pharmaceutical composition according to the present invention may increase the Th1 response and decrease the Th2 response. The use of an immune modulator composition and/or pharmaceutical composition according to the present invention may increase the Th1 response and increase the Th2 response. A skilled person can test a specific species of each genus according to the present invention to determine its specific Th1/Th2 response. An unregulated or inappropriately regulated immune response may play a role in the establishment of disease due to the fact that some diseases cause, or are a consequence of, shifted Th1 and/or Th2 responses. Accompanying these atypical Th1 and Th2 reactions are a series of abnormal inflammatory responses, which may take part in the mechanisms underlying tissue pathology. By way of example only, the immune modulator composition and/or pharmaceutical composition according to the present invention may counteract an autoimmune disease or autoimmune disorder.

### Vaccines

The preparation of vaccines which contain one or more substances as an active ingredient(s), is known to one skilled in the art. Typically, such vaccines are prepared as injectables, either as liquid solutions or suspensions; solid forms suitable for solution in, or suspension in, liquid prior to injection may also be prepared. The preparation may also be emulsified, or the active ingredient(s) encapsulated in liposomes. The active ingredients are often mixed with excipients which are pharmaceutically acceptable and compatible with the active ingredient. Suitable excipients are, for example, water, saline, dextrose, glycerol, ethanol, or the like and combinations thereof. Alternatively, the vaccine may be prepared, for example, to be orally ingested and/or capable of inhalation. In addition, if desired, the vaccine may contain minor amounts of auxiliary substances such as wetting or emulsifying agents and pH buffering agents.

### Administration

Typically, a physician will determine the actual dosage of a vaccine, immune modulator composition and pharmaceutical composition which will be most suitable for an individual subject and it will vary with the age, weight and response of the particular patient. The dosages below are exemplary of the average case. There can, of course, be individual instances where higher or lower dosage ranges are merited. Preferably, the actual dosage that is used results in minimal toxicity to the subject. The compositions of the present invention may be administered by direct injection. The composition may be formulated for parenteral, mucosal, intramuscular, intravenous, subcutaneous, intraocular, intradermal or transdermal administration. The composition according to the present invention may be administered at a dose of 1 nanogram to 100 milligrams organisms, preferably 10 nanograms to 10 milligrams organisms, more preferably 100 nanograms to 5 milligrams organisms, and even more preferably 100 nanograms to 1 milligram organisms. Typically, the composition according to the present invention may be administered at a dose of 100 micrograms to 1 milligram bacteria for human and animal use.

If the compositions of the present invention are to be administrated as immune enhancers, then 1 nanogram to 100 milligrams organisms per dose, preferably 10 nanograms to 10 milligrams organisms per dose, more preferably 100 nanograms to 5 milligrams organisms per dose, and even more preferably 100 nanograms to 1 milligram organisms per dose, and even more preferably, 100 micrograms to 1 milligram bacteria per dose for human and animal use may be administered at regular intervals. As will be readily appreciated by a skilled person the dosage administered will be dependent upon the organism to which the dose is being administered.

The term “administered” includes delivery by delivery mechanisms including injection, lipid mediated transfection, liposomes, immunoliposomes, lipofectin, cationic facial amphiphiles (CFAs) and combinations thereof, or even viral delivery. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestable solution, capsule or tablet; a parental route where delivery is by an injectable form, such as, for example, an intravenous, intramuscular, intradermal or subcutaneous route.

The term “co-administered” means that the site and time of administration of each of the adjuvants(s), antigen(s) and/or antigenic determinant(s) of the present invention are such that the necessary modulation of the immune system is achieved. Thus, whilst the antigen(s) and adjuvant(s) may be administered at the same moment in time and at the same site, there may be advantages in administering the antigen(s) and/or antigenic determinant(s) at a different time and to a different site from the adjuvant(s). The antigen(s) and/or antigenic determinant(s) and adjuvant(s) may even be delivered in the same delivery vehicle—and the antigen(s) and/or antigenic determinant(s) and adjuvant(s) may be coupled and/or uncoupled and/or genetically coupled and/or uncoupled. By way of example only, the immune modulator composition according to the present invention may be administered before, at the same time or post administration of one or more antigens or further antigens.

The antigen, antigenic determinant, peptide or homologue or mimetic thereof may be administered separately or co-administered to the host subject as a single dose or in multiple doses.

The immune modulator composition and/or pharmaceutical composition of the invention may be administered by a number of different routes such as injection (which includes parenteral, subcutaneous, intradermal and intramuscular injection) intranasal, mucosal, oral, intra-vaginal, urethral or ocular administration. Preferably, in the present invention, administration is by injection. More preferably the injection is intradermal. Preferably, in the present invention, administration is by an orally acceptable composition.

For vaccination the composition can be provided in 0.1 to 0.2 ml of aqueous solution, preferably buffered physiological saline, and administered parenterally, for example by intradermal inoculation. The vaccine according to the invention is preferably injected intradermally. Slight swelling and redness, sometimes also itching may be found at the injection site. The mode of administration, the dose and the number of administrations can be optimised by those skilled in the art in a known manner.

### Antigens

As used herein, an “antigen” means an entity which, when introduced into an immunocompetent host, modifies the production of a specific antibody or antibodies that can combine with the entity, and/or modifies the relevant Th response, such as Th2 and/or Th1, The antigen may be a pure substance, a mixture of substances or soluble or particulate material (including cells or cell fragments or cell sonicate). In this sense, the term includes any suitable antigenic determinant, cross reacting antigen, alloantigen, xenoantigen, tolerogen, allergen, hapten, and immunogen, or parts thereof, as well as any combination thereof, and these terms are used interchangeably throughout the text.

The term “antigenic determinant or epitope” as used herein refers to a site on an antigen which is recognised by an antibody or T-cell receptor, or is responsible for evoking the T-helper cell response. Preferably it is a short peptide derived from or as part of a protein antigen. However the term is also intended to include glycopeptides and carbohydrate epitopes. The term also includes modified sequences of amino acids or carbohydrates which stimulate responses which recognise the whole organism.

A “preventative” or “prophylactic” vaccine is a vaccine which is administered to naive individuals to prevent development of a condition, such as by stimulating protective immunity.

A “therapeutic” vaccine is a vaccine which is administered to individuals with an existing condition to reduce or minimise the condition or to abrogate the immunopathological consequences of the condition.

### Adjuvants

The term ‘adjuvant’ as used herein means an entity capable of augmenting or participating in the influencing of an immune response. An adjuvant is any substance or mixture of substances that assists, increases, downregulates, modifies or diversifies the immune response to an antigen.

The immune modulator composition and/or pharmaceutical composition according to the present invention may comprise one or more adjuvants which enhance the effectiveness of the immune modulator composition and/or pharmaceutical compositions. Examples of additional adjuvants which, may be effective include but are not limited to: aluminium hydroxide, aluminium phosphate, aluminium potassium sulphate (alum), beryllium sulphate, silica, kaolin, carbon, water-in-oil emulsions, oil-in-water emulsions, muramyl dipeptide, bacterial endotoxin, lipid X, Corynebacterium parvum (Propionobacterium acnes), Bordetella pertussis, Mycobacterium vaccae, polyribonucleotides, sodium alginate, lanolin, lysolecithin, vitamin A, interleukins such as interleukin 2 and interleukin-12, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.). Only aluminium hydroxide is approved for human use. Some of the other adjuvants, such as M. vaccae for example, have been approved for clinical trials. Suitably, the adjuvant may be humic substances.

In the art, it is known that DNA vaccines, which are essentially DNA sequences attached to gold particles and which are fired into the skin by a helium gun, are efficient vaccine delivery systems. Unlike conventional vaccines, these DNA vaccines do not require a traditional adjuvant component. In accordance with a further aspect of the present invention, the immune modulator composition as defined herein may suitably be used in conjunction with such DNA vaccines to augment or participate in the influencing of the immune response.

### Pharmaceutical Compositions

The present invention also relates to a pharmaceutical composition comprising a therapeutically effective amount of Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts and optionally a pharmaceutically acceptable carrier, diluent or excipients (including combinations thereof).

The pharmaceutical composition may comprise two components—a first component comprising an antigen and a second component comprising an adjuvant thereof. The first and second component may be delivered sequentially, simultaneously or together, and even by different administration routes.

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice.

The pharmaceutical compositions may comprise as—or in addition to—the carrier, excipient or diluent any suitable binder(s), lubricant(s), suspending agent(s), coating agent(s), solubilizing agent(s).

Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular, intradermal or subcutaneous route. Alternatively, the formulation may be designed to be delivered by both routes.

Preferably in the present invention the formulation is of injectable form. More preferably the formulation is intradermally injected.

Preferably in the present invention the formulation is an orally acceptable composition.

Where the agent is to be delivered mucosally through the gastrointestinal mucosa, it should be able to remain stable during transit through the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly, intradermally or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

### Pharmaceutical Combinations

The agent of the present invention may be administered with one or more other pharmaceutically active substances. By way of example, the present invention covers the simultaneous, or sequential treatments with an immune modulator composition and/or pharmaceutical composition according to the present invention, and one or more steroids, analgesics, antivirals, interleukins such as IL-2, or other pharmaceutically active substance(s). It will be understood that these regimes include the administration of the substances sequentially, simultaneously or together.

### Immune Enhancer

The term “immune enhancer” as used herein means one or more bacteria either isolated or in culture which when administered to a subject benefit the health of that subject. Preferably, this benefit is achieved by the modification of the cellular immune response of the subject.

In accordance with the present invention, immune enhancers may be used, for example, for the treatment or prevention of an autoimmune disease or autoimmune disorder. The immune enhancers may be administered by consumption in specially designed food or in animal feeds. The immune enhancers may also be administered by other routes—such as direct injection.

Identifying Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts that Modulate a Cellular Immune Response

In another aspect, the present invention relates to a method for identifying one or more whole humic substances that modulate (e.g. modify) a cellular immune response comprising the steps of: (a) contacting a first test animal with an immunostimulant; (b) contacting a second test animal with an immunostimulant mixed with humic substances; (c) measuring the cellular immune response in each of the test animals; and (d) comparing the cellular immune response in each of the test animals, wherein, a lower cellular immune response from the immunostimulant mixed with humic substances in comparison to the immunostimulant alone is indicative of a modification of the cellular immune response by humic substances.

In another aspect, the present invention relates to a method of determining the Th1/Th2 response of Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts substances which method comprises utilisation of the tuberculin skin test. In mice, the tuberculin skin test is preferably carried out on the foot pad. In a predominant Th1 reaction the positive foot pad immune response is maximal at 24 hours and diminishes at 48 hours. However, as the Th2 reactivity increases then the 48 hour positive foot pad immune response increases and can even exceed the foot pad immune response at 24 hour.

The effect of BCG vaccination is well documented using this tuberculin skin test. Thus, the test assay can be used to assess whether or not the introduction of an immune modulator composition according to the present invention modulates the BCG cellular immune response.

As used herein, the term “test animal” refers to any animal that elicits a cellular immune response to the immunostimulant. Preferably, the test animal(s) is a mammal. More preferably, the test animal(s) is a rat, hamster, rabbit, guinea pig or mouse. More preferably, the test animal(s) is a mouse.

Preferably, Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts modify the T helper cell response. Suitably, humic substances may modify the T helper cell response by decreasing the Th1 and Th2 response. Suitably, humic substances may modify the T helper cell response by increasing the Th1 response and decreasing the Th2 response. Suitably, humic substances may modify the T helper cell response by increasing the Th1 response without affecting the Th2 response.

Preferably, the immunostimulant will have a known Th1 and Th2 response. For example, with the immunostimulant BCG the reaction is usually largest at 24 h when it is an indicator of the Th1 response; the reaction at 48 h is usually less and includes a Th2 contribution. It is known that BCG predominantly stimulates a Th1 response. By use of such immunostimulants it may be possible to determine the Th1/Th2 response of a test bacterium and, thus, it may be possible to identify humic substances which have a desired Th1/Th2 response to treat and/or prevent a particular disease and/or disorder.

Preferably, the cellular immune response is measured using the tuberculin skin test. Vaccination with an immunostimulant—such as BCG—induces a response to skin-testing with tuberculin (a soluble preparation of Tubercle bacilli), when tested later. The local reaction is measured at various intervals, for example, 24 hours, 48 hours and 72 hours after injection of tuberculin. Briefly, immunostimulants (Immunity-inducing a combination guanidinium derivatives, particularly to combinations of oligo(2-(2-ethoxy)ethoxy ethyl guanidinium chloride), poly(hexamethylendiamine guanidinium chloride), mushroom, enzymes, PGPR, amino acids, antioxidants like humic acids and some natural products like phytotherapeutic plant extracts substances) are used that induce a positive immune response to tuberculin. In the test animal, the tuberculin skin test is preferably carried out on the foot pad. In a predominant Th1 reaction the positive foot pad immune response is usually maximal at 24 hours and diminishes at 48 hours. However, as the Th2 reactivity increases then the 48 hour positive foot pad immune response increases and can even exceed the foot pad immune response at 24 hour. Thus, the assay can be used to assess whether or not the introduction of an immune modulator composition according to the present invention modulates the cellular immune response.

The present invention provides methods for increasing the therapeutic index of cancer therapeutic compounds used in the treatment of cancer. The present invention also provides methods for increasing the therapeutic index of antiviral agents used in the treatment of antiviral diseases. More specifically, the present invention provides such methods which include administering one or more anticancer or antiviral agent in combination with a therapeutically effective amount of a composition comprising a pharmaceutically acceptable carrier and material or chemical from, or herbal preparation comprising a plant species of each of the following genera of herbs: Scutellaria, Glycyrrhiza, Ziziphus and Paeonia. The methods of the present invention provide the use of material or chemical from, or herbal preparation comprising such herbs which is in the form of a granulated extract from an aqueous solution that includes but is not limited to water and alcohol. Such compositions can be in an ingestible form, such as, but not limited to, powders, capsules, liquids and tablets. Alternatively, the methods of the present invention use such compositions in the form of a suppository.

The present invention also provides methods of treating diseases in mammals in need of such treatment which includes administering a therapeutically effective amount of a composition comprising a pharmaceutically acceptable carrier; material or chemical from, or herbal preparation comprising a plant species of each of the following genera of herbs: Scutellaria, Glycyrrhiza, Ziziphus and Paeonia; and one or more chemotherapeutic compounds.

The present invention further provides methods of treating diseases in a mammal in need of such treatment which includes administering a therapeutically effective amount of one or more chemotherapeutic compounds or antiviral agents and a composition which includes a pharmaceutically acceptable carrier; material or chemical from, or herbal preparation comprising a plant species of each of the following genera of herbs: Scutellaria, Glycyrrhiza, Ziziphus and Paeonia. The present invention includes such methods wherein the composition is administered before the administration of the one or more chemotherapeutic compounds. The present invention also includes such methods wherein the composition is administered after the administration of the one or more chemotherapeutic compounds.

The present invention provides methods of modulating hematopoietic activity for the treatment of a disease by administering to a mammal in need of such treatment a therapeutically effective amount of a composition consisting essentially of a pharmaceutically acceptable carrier and material or chemical from or herbal preparation comprising a plant species of each of the following genera of herbs: Scutellaria, Glycyrrhiza, Ziziphus and Paeonia. The present invention provides such methods wherein the material or chemical from the herbs is in the form of a granulated extract from an aqueous solution that includes but is not limited to water and alcohol. Specifically, the present invention provides such methods wherein the composition is in an ingestible form, such as, but not limited to, powders, capsules, liquids and tablets. Alternatively, the present invention provides such methods wherein the composition is in the form of a suppository.

1. A method for treating an autoimmune disease or an autoimmune disorder comprising administering an effective amount of a composition comprising Guanidinium Derivatives to a subject wherein said composition modulates a cellular immune response, and wherein the autoimmune disease or autoimmune disorder involves inflammation of the intima of a blood vessel and is a vascular disorder selected from the group consisting of atheroma formation (otherwise known as arteriosclerosis), myointimal hyperplasia (natural or following angioplasty), inflammatory and autoimmune thickening of the intema and/or muscular layer of blood vessels and myocarditis.

2. A method for immune modulation of a subject against an autoimmune disease or an autoimmune disorder comprising administering a composition comprising Guanidinium Derivatives wherein the autoimmune disease or autoimmune disorder involves inflammation of the intima of a blood vessel and is a vascular disorder selected from the group consisting of atheroma formation (otherwise known as arteriosclerosis), myointimal hyperplasia (natural or following angioplasty), inflammatory and autoimmune thickening of the intima and/or muscular layer of blood vessels and myocarditis. Immunity-inducing agent composition characterised in that it contains at least 500 mg/l, preferably at least 1000 mg/l, more preferably at least 600 mg/l, even of a combination guanidinium derivatives, particularly to combinations of oligo(2-(2-ethoxy)ethoxy ethyl guanidinium chloride), poly(hexamethylendiamine guanidinium chloride), mushrooms, enzymes, PGPR, amino acids, antioxidants such as humic acids and some natural products like phytotherapeutic plant extracts.

3. The composition of Immunity-Inducing Agent product by produce the methods according to claim 1 or 2, wherein the guaninidium compound is oligo(2-(2-ethoxy)ethoxy ethyl guanidinium chloride).

4. The composition of Immunity-Inducing Agent product by produce the methods according to claim 1 or 2, wherein the guaninidium compound is poly(hexamethylendiamine guanidiniumchloride).

5. Immunity-Inducing Agent composition by produce the methods according to claim 1 or 2, characterized in that it has a pH of between 5.5 and 7.5.

6. Immunity-Inducing Agent composition by produce the methods according to claim 1 or 2 characterized in that it is selected within the group consisting in human and animal.

7. Immunity-Inducing Agent composition by produce the methods according to claim 1 or 2 where in a dosage form of the immunity-inducing agent in is soft gel or capsulation

8. Immunity-Inducing Agent composition by produce the methods according to claim 1 or 2 characterized in that it is useful for therapy or prophylaxis of cancer.

9. Immunity-Inducing Agent composition by produce the methods according to claim 1 or 2 characterized in that it is useful for therapy for breast cancer.

10. Immunity-Inducing Agent composition by produce the methods according to claim 1 or 2 characterized in that it is useful for therapy brain tumor,

11. Immunity-inducing agent composition by produce the methods according to claim 1 or 2 characterized in that it is useful for therapy esophagus cancer.

12. Immunity-Inducing Agent composition by produce the methods according to claim 1 or 2 characterized in that it is useful for therapy lung cancer.

13. Immunity-inducing agent composition according to claim 1 or 2 characterized in that it is useful for therapy renal cancer.

14. Immunity-inducing agent composition by produce the methods according to claim 1 or 2 characterized in that it is useful for therapy colon cancer.

15. Immunity-inducing agent composition by produce the methods according to claim 1 or 2 characterized in that it is useful for therapy lever cancer.

16. Immunity-inducing agent composition by produce the methods according to claim 1 or 2, characterized in that it is useful for therapy prostate cancer.

17. Immunity-inducing agent composition by produce the methods according to claim 1 or 2, characterized in that it is useful for therapy thyroid cancer.

18. The composition of Immunity-Inducing Agent by produce the methods claim 1 or 2, wherein the Immunity-inducing agent composition is a PGPR, Plant Growth Promoting Rhizobacteria, said biological agent is an antibody selected from the group consisting of Acinetobacter, Achromobacter, Aereobacter, Agrobacterium, Alcaligenes, Artrobacter, Azospirillum, Serratia, Bacillus, Burkholderia, Enterobacter, Erwinia, Flavobacterium, Microccocus, Pseudomonas, Rhizobium ve Xanthomonas.

19. The composition of Immunity-Inducing Agent by produce the methods according to claim 1 or 2, wherein the antioxidant is humic acid leached from leonardite ore and its sodium/potassium salts.

20. The composition of Immunity-Inducing Agent by produce the methods according to claim 1 or 2, wherein amino acids are from a group of L-cysteine, and L-arginine.

21. The composition of Immunity-Inducing Agent by produce the methods according to claim 1 or 2, wherein enzymes are from a group glutaminase, Arginine decarboxylase, histidine decarboxylase (Lactobacillus), and carboxypeptidase.

22. The composition of Immunity-Inducing Agent by produce the methods according to claim 1 or 2, wherein mushrooms are from a group Lentinus edodes, Ganoderma lucidum, Pleurotus spp, Hericium erinaceus, Coriolus versicolor, Auricularia spp.

23. The method of claim 1 or 2, wherein herbal plants are Aniseed (Anisi fructus), Barbados Aloes (Aloe barbadensis), Bearberry leaf (Uvae ursi folium), Bilberry Fruit (Myrtilli fructus), Birch Leaf (Betulae folium), Black Cohosh (Cimicifugae rhizoma), Black Currant Leaf (Ribis nigri folium), Black Horehound (Ballotae nigrae herba), Bogbean leaf (Menyanthidis trifoliatae folium), Burdock Root (Arctii radix), Butcher's Broom (Rusci rhizome), Cape Aloes (Aloe capensis), Cascara (Rhamni purshianae cortex), Centaury (Centaurii herba), Clove oil (Caryophylli aetheroleum), Cola (Colae semen), Comfrey root (Symphyti radix), Couch Grass Rhizome (Graminis rhizoma), Elder flower (Sambuci flos), Feverfew (Tanaceti parthenii herba), Frangula Bark (Frangulae cortex), Gentian Root (Gentianae radix), Grindelia (Grindeliae herba), Hamamelis bark (Hamamelidis cortex), Hamamelis leaf (Hamamelidis folium), Hamamelis water (Hamamelidis aqua), Hydrastis rhizoma (Goldenseal rhizome), Ispaghula Husk (Plantaginis ovatae testa), Java Tea (Orthosiphonis folium), Lady's Mantle (Alchemillae herba), Linseed (Lini semen), Mallow Flower (Malvae flos), Meadowsweet (Filipendulae ulmariae herba), Melissa leaf (Melissae folium), Myrrh (Myrrha), Mullein flower (Verbasci flos), Nettle Root (Urticae radix), Pelargonium Root (Pelargonii radix), Psyllium Seed (Psylli semen), Restharrow Root (Ononidis radix), Rhatany Root (Ratanhiae radix), Ribwort Plantain leaf/herb (Plantaginis lanceolatae folium/herba), Sage Leaf, Trilobed (Salviae trilobae folium), Tormentil (Tormentillae rhizoma), White Horehound (Marrubii herbal), Wild Pansy (Violae herba cum flore), Wild Thyme (Serpylli herba), Willow Bark (Salicis cortex).

24. The composition of Immunity-Inducing Agent by produce the methods according claim 1 or 2, wherein it contains cephamine and dopamine.

25. The composition Immunity-Inducing Agent by produce the methods according of claim 1 or 2, wherein it contains cyclic oligosaccharide, preferably β-cyclodextrin or γ-cyclodextrin.

26. The composition of Immunity-Inducing Agent by produce the methods according claim 1 or 2, wherein it contains the vitamins or a group of them, preferably Vitamin A, C, and D.

27. Immunity-inducing agent composition by produce the methods according according to claim 1 or 2 characterized in that it is an immunoenhancer.
